(19) United States
(12) Patent Application Publication
(10) Pub. No.: US 2003/0191404 A1
(43) Pub. Date: Oct. 9, 2003

(54) Method and apparatus for providing arrhythmia discrimination

(76) Inventor: George J. Klein, London (CA)

Correspondence Address: ORANGE & CHARI, Suite 4900, P.O. Box 190, 66 Wellington St. W., Toronto, ON, M5K 1H6, CA

(21) Appl. No.: 10/117,187

(22) Filed: Apr. 8, 2002

(51) Int. Cl. A61N 1/362

(52) U.S. Cl. 600/518; 607/14; 607/3

(57) ABSTRACT

The present invention provides a method and apparatus for providing arrhythmia discrimination. Heart signals are monitored to determine when an arrhythmia state is being experienced by an individual. Once the arrhythmia state is detected, the heart of the patient is stimulated to produce stimulated heart signals. These stimulated signals are then monitored and compared with predetermined criteria in order to determine the type of arrhythmia being experienced as well as whether or not the arrhythmia requires treatment.

### BACKGROUND OF THE INVENTION

1. Filed of the Invention

The present invention is directed to medical treatment devices and more specifically to a method and apparatus for providing arrhythmia discrimination.

2. Description of the Prior Art

In the field of arrhythmia treatment devices such as defibrillators, pacemakers and the like, recipients of such devices are dependent on their proper functioning for treatment of life-threatening cardiac arrhythmias. An arrhythmia is seen as any deviation from or disturbance of a normal heart rhythm. This includes an acceleration or a deceleration from the normal heart rhythm. Although known arrhythmia treatment devices attempt to discriminate between life-threatening arrhythmias, i.e. “target” arrhythmias, and those which are not life-threatening, they sometimes misclassify arrhythmias that are, in fact, benign as “target” arrhythmias. This results in inappropriate treatment being administered to the patient, generally in the form of cardioversion or “shock” therapy which is quite painful to patients. However, adjustment of the devices, by lowering a detection threshold, to minimize inappropriate treatment entails a risk of the treatment device not detecting the life-threatening target arrhythmias, which may possibly result in the death of the patient.

Current arrhythmia treatment devices, such as implantable defibrillators, execute a detection process to monitor and analyze heart rate signals or electrocardiograms to determine when the heart rate of the patient is greater than a predetermined threshold such that arrhythmia, in the form of a tachycardia, is detected. Some prior art defibrillators such as U.S. Pat. No. 6,076,014, may also use the relative timing of heart signals from different locations within the heart such as the atrium or the ventricle to assist in the detection of other types of arrhythmia. After the arrhythmia has been detected, treatment necessary to treat the detected type of arrhythmia is administered to the patient. If an appropriate treatment cannot be determined for the detected arrhythmia, the device administers a default treatment to treat the arrhythmia in order to avoid missing a life-threatening situation. In some cases, the arrhythmia is not life-threatening and therefore the patient will receive inappropriate treatment which results in pain and discomfort.

It is an object of the present invention to obviate and mitigate the above disadvantages.

### SUMMARY OF THE INVENTION

In an aspect of the present invention, there is provided a method of providing arrhythmia discrimination comprising the steps of receiving heart signals, representing a heart rate, from a heart; monitoring the heart signals to detect an arrhythmia state; detecting the arrhythmia state; performing a diagnostic test on the heart to produce stimulated heart signals; and monitoring the stimulated heart signals to determine the nature of the arrhythmia.

In another aspect, there is provided apparatus for providing arrhythmia discrimination comprising a set of electrodes for sensing and transmitting heart signals from a heart; a heart signal monitor for receiving the heart signals and for translating the heart signals to processor-readable heart signals; a processor for retrieving rate signals from the processor-readable heart signals to detect an arrhythmia state based on the rate signals and for selecting a diagnostic test when the arrhythmia is detected; wherein the diagnostic test causes the heart to produce stimulated heart signals which are sensed and transmitted by the set of electrodes to the heart signal monitor for translating to processor-readable stimulated heart signals; and wherein the processor retrieves stimulated rate signals from the processor-readable stimulated heart signals and compares the stimulated rate signals with predetermined criteria to discriminate the arrhythmia.

### BRIEF DESCRIPTION OF THE DRAWINGS

These and other features of the preferred embodiments of the invention will become more apparent in the following detailed description in which reference is made to the appended drawings wherein:

FIG. 1 is a schematic diagram of an embodiment of apparatus for providing arrhythmia discrimination;

FIG. 2 is a enlarged view of a processor and a database for use in providing arrhythmia discrimination;

FIG. 3 is a flowchart outlining a first mode of operation of the apparatus of FIGS. 1 and 2;

FIG. 4 is a flowchart outlining in further detail the mode of operation of the apparatus of FIGS. 1 and 2;

FIG. 5a and 5b are schematic views of intracardiac heart signals;

FIG. 6 is a schematic view of a cardiac cycle; and

FIG. 7 is a schematic view of a set of cardiac cycles.

### DESCRIPTION OF THE PREFERRED EMBODIMENTS

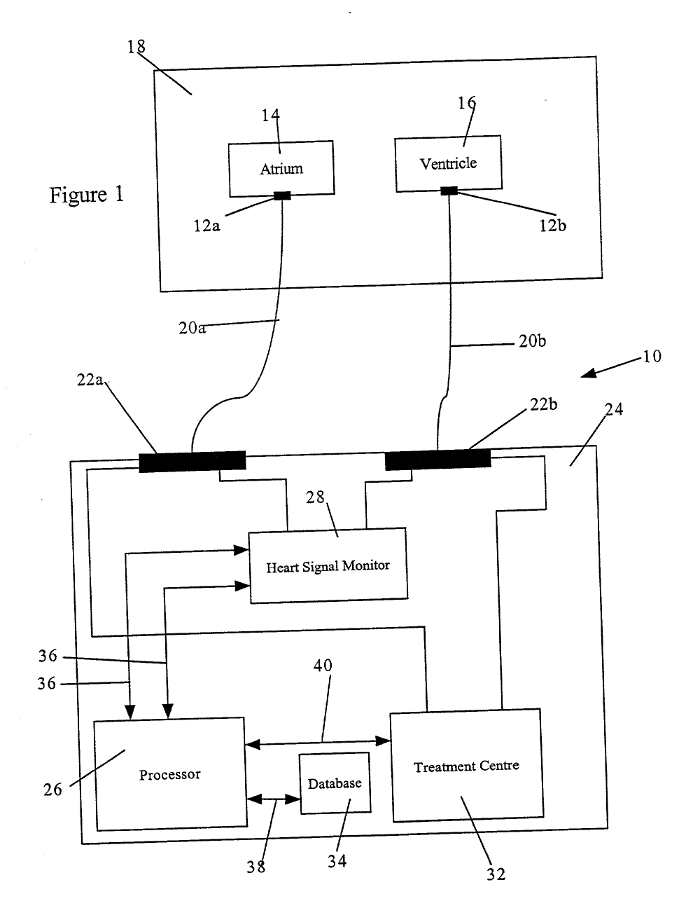

Turning to FIG. 1, a schematic diagram of an embodiment of the apparatus for providing arrhythmia discrimination is shown. The apparatus 10 includes a set of electrodes 12 (seen as atrial electrode 12a and ventricular electrode 12b) which are preferably located on the endocardium or epicardium of the atrium 14 and ventricle 16 of a patient's heart 18. A set of leads 20a and 20b connect the electrodes 12 to I/O ports 22 (seen as I/O ports 22a and 22b) located within a housing 24. The housing 24 further includes a processor 26, a heart signal monitor 28 to receive and compile signals from electrodes 12 and transmit them to processor 26 over communication bus 36. The housing 22 also includes a treatment centre 32, in communication with the processor 26 via communication bus 40, to deliver a treatment protocol to the heart through the electrodes 12. The housing 22 may also include a database 34 which stores a list of diagnostic tests along with a list of appropriate treatments corresponding to detected types of arrhythmia. These lists are accessed by the processor 26 via communication bus 38.

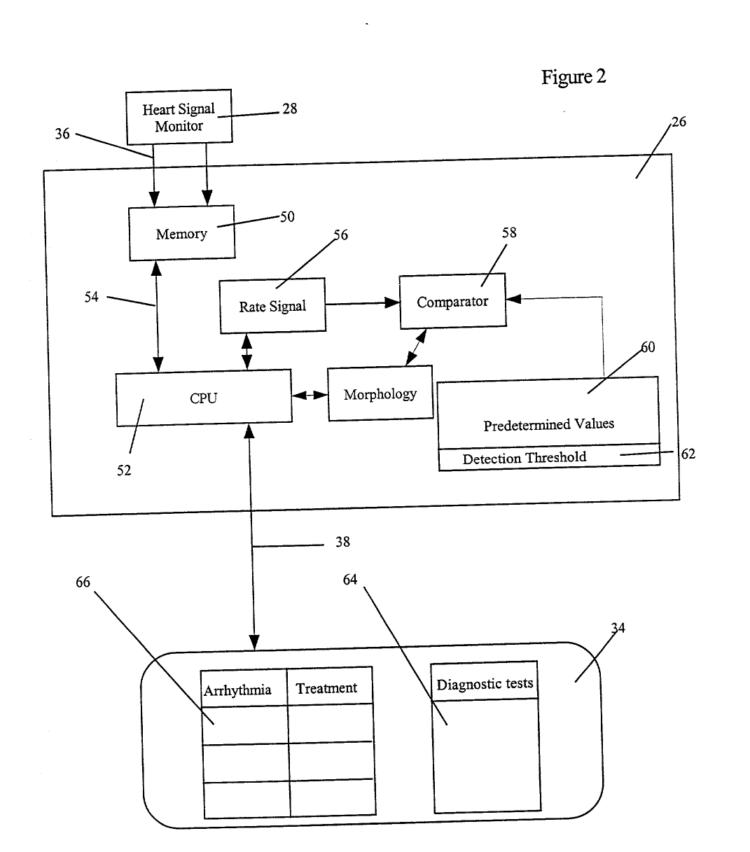

The processor 26 is shown in greater detail in FIG. 2. The processor 26 includes memory 50, preferably in the form of First-in-First-out (FIFO) memory, to store the signals which are received from the heart signal monitor 28. A central processing unit (CPU) 52 accesses the memory 50 over communication bus 54 to retrieve the stored signals and to calculate rate signals corresponding to the stored signals. After the rate signals are retrieved, they are stored in a rate memory location 56. The CPU 52 may also retrieve morphology signals from the stored signals representing the shape of the heart signals which are then stored in a morphology memory location 57. The rate signals are then transmitted to a comparator 58 which compares the rate signals with predetermined stored values 60, such as a pre-stored detection threshold 62. This will be described in more detail below. Once an arrhythmia state has been detected, the CPU 52 accesses the database 34 via communication bus 38, to access a diagnostic test from the list of diagnostic tests 64. In order to determine the appropriate treatment for a type of arrhythmia, the CPU 52 accesses a treatment look-up table 66 within the database 34. The look-up table 66 preferably includes a listing of types of arrhythmia along with their corresponding treatment.

While U.S. Pat. 6,076,014 discloses one method of implanting the electrodes 12 and leads 20 within a patient's heart, other methods of implanting the electrodes 12 will be apparent to persons skilled in the art.

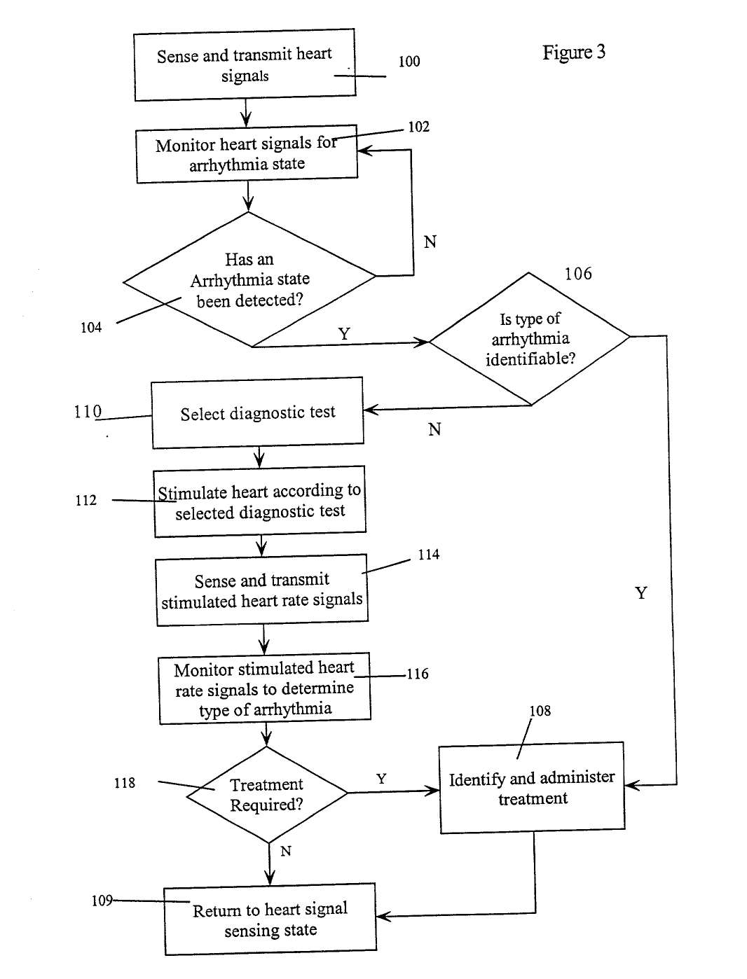

In general terms, the apparatus 10 functions as shown schematically in FIG. 3. The electrodes 12 sense heart signals from their location within the heart 18 and transmit these heart signals to the I/O ports 22 (step 100). The atrial electrode 12a and the ventricular electrode 12b each transmit a set of heart signals to the heart signal monitor 28. It will be understood that although two electrodes are shown in the present embodiment, the number of electrodes 12 used may vary. Furthermore, the location of the electrodes 12 may also be varied within the patient's heart 18 provided that the selected location provides heart signals to be sensed.

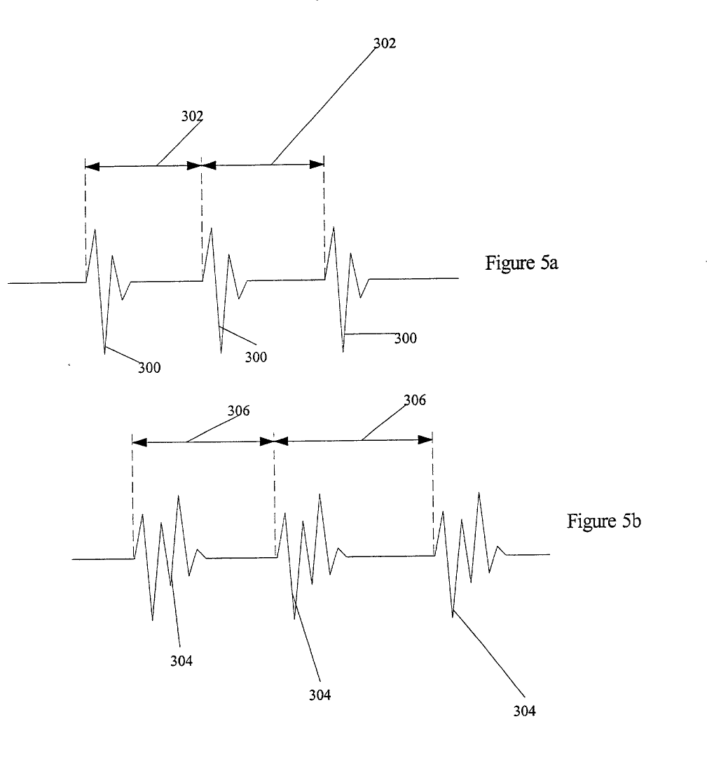

After receiving the heart signals, the I/O ports 22 transmit the signals to the heart signal monitor which translates the heart signals into a format which is readable by the processor 26 such as shown in FIGS. 5a and 5b with FIG. 5a showing heart signals received from the atrial electrode 12a and FIG. 5b showing heart signals received from the ventricular electrode 12b.

The formatted signals are then transmitted to the processor 26 via communication buses 36 and stored in memory 50. The CPU 52 then accesses the signals and retrieves information such as rate signals from the formatted signals and extracts information from the rate signals to detect an arrhythmia state (step 102). The information and the rate signals are then stored in the rate memory location 56. The rate signals and the detection threshold 62 are then sent to the comparator 58 for comparison with each other to determine when an arrhythmia state is being experienced by the heart (step 104). The detection threshold 62 is a predetermined value which is pre-stored within the processor 26 and represents a value which the heart rate is not expected to exceed.

If the rate signals are lower than the detection threshold, the processor 26 simply receives the next set of formatted signals from the heart signal monitor 28 and stores them in the memory 50 before the CPU 52 accesses the signals to retrieve the rate signals for comparison with the detection threshold 62 to determine if an arrhythmia state is being experienced. However, if the rate signals are higher than the detection threshold, a check is performed to see whether the type of arrhythmia being experienced may be identified by the information contained in the rate signals (step 106). If the type of arrhythmia may be determined from the information in the rate signals, the appropriate treatment is identified by the processor 26. The processor 26 identifies the appropriate treatment by accessing the treatment look-up table 66 in the database 34. After retrieving the type of treatment, the processor 26 signals, via communication bus 40, the treatment protocol to the treatment centre 32 which then sends out signals instructing the electrodes 12 to administer the treatment protocol, such as cardioversion therapy, to the heart (step 108). After administering the treatment, the electrodes 12 return to sensing and transmitting heart signals (step 109).

If the type of arrhythmia is not determinable, or ambiguous, the processor 26 selects a diagnostic test (step 110) from the list of diagnostic tests 64 stored in the database 34. The diagnostic test is used to produce stimulated heart signals which are monitored to determine the type of arrhythmia being experienced and is preferably based on the rate signals of the atrium or ventricle when the arrhythmia was detected.

After the diagnostic test has been selected by the processor 26, the processor 26 communicates with the treatment centre 32 to stimulate the heart according to the selected diagnostic test (step 112). The treatment centre 32 communicates with the electrodes 12 to administer the stimulation, which is preferably achieved via a pacing of the heart. The stimulation may be performed by only a select number of electrodes 12 and does not have to be performed by each electrode 12. In this manner, some electrodes 12 may continue to sense and transmit heart signals to the I/O ports 22.

After the heart has been stimulated, the electrodes 12 sense the heart signals (seen as stimulated heart signals) and transmit them to the I/O ports 22 (step 114) which, in turn, transmit them to the heart signal monitor 28. As with heart signals, the heart signal monitor 28 translates the stimulated heart signals into a format readable by the processor 26 and transmits the formatted stimulated heart signals to the processor 26 for storage in the memory 50.

The CPU 52 then accesses the formatted signals to retrieve information such as stimulated rate signals and morphology signals and extracts information from the stimulated rate signals to determine the type of arrhythmia being experienced (step 116) by comparing the stimulated rate signals with predetermined criteria. After determining the type of arrhythmia, the processor 26 determines whether or not treatment is required for the identified arrhythmia (step 118). If treatment is required, it is identified and administered (step 108), in the manner described above. Examples of arrhythmia which require treatment include supraventricular tachycardias such as atrial flutter, atrial fibrillation, ectopic atrial tachycardia, atrioventricular junctional re-entry, ventricular tachycardias and ventricular fibrillation. In addition, there may be two or more of the above target arrhythmias simultaneously present (double tachycardias or triple tachycardias etc). After administering the treatment, the electrodes 12 continue to sense and transmit heart signals to the I/O ports 22 (step 109).

If treatment is not required, the electrodes 12 continue to sense and transmit heart signals to the I/O ports 22 (step 109).

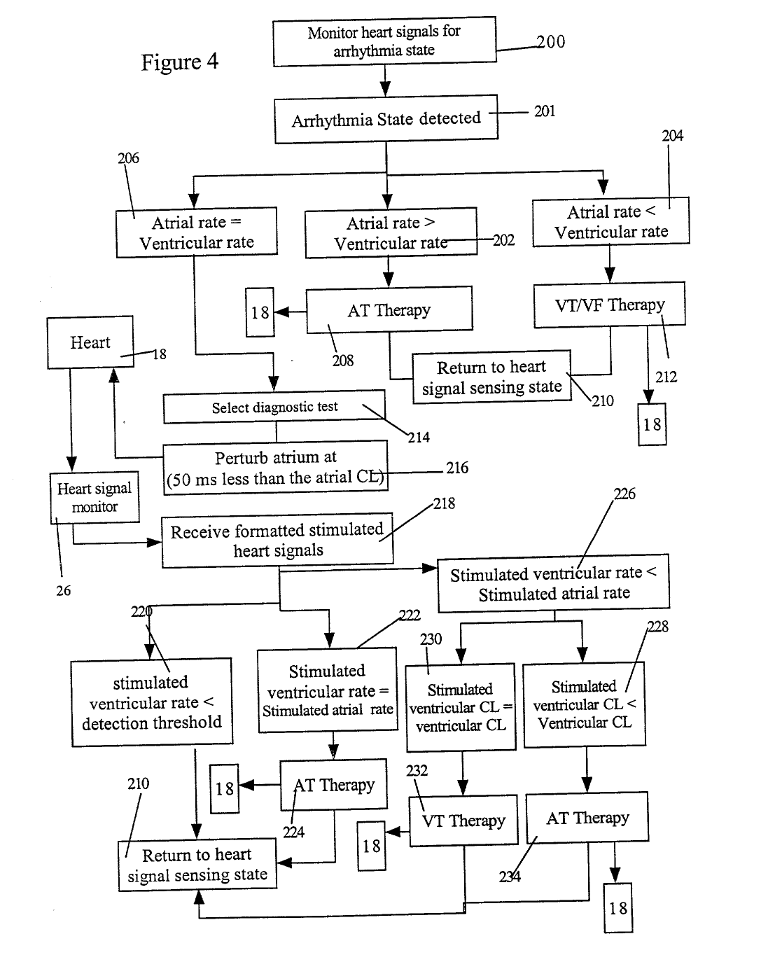

The detailed operation of the apparatus 10 is exemplified in FIG. 4 using two electrodes (atrial electrode 12a and ventricular electrode 12b), respectively connected to the atrium and ventricle of a patient's heart.

As described with respect to FIG. 3, heart signals are monitored to determine when an arrhythmia state is being experienced (step 200). The heart signals are sensed by the electrodes 12 and transmitted to the heart signal monitor 28 via the I/O ports 22. The heart signals are then translated by the heart signal monitor 28 into a format which is readable by the processor 28 such as the electrograms shown in FIGS. 5a and 5b for the atrium and ventricle respectively.

After receiving the formatted signals and storing the signals in memory 50, the CPU 52 retrieves the cycle length of the heart signals from the atrial electrode 12a and the ventricular electrode 12b by calculating the time elapsed between successive atrial electrograms 300 to determine an atrial cycle length 302 along with the time elapsed between successive ventricular electrograms 304 to determine a ventricular cycle length 306. Rate signals, representing an inverse of the cycle length, are then calculated and stored in the rate memory location 56. Along with the rate signals, morphology signals representing the shape of the heart signals may also be retrieved and stored in the morphology memory location 57. The rate signals corresponding to the atrial and ventricular rates are then compared, using the comparator 58, to the pre-stored detection threshold 62 in order to determine if the heart is experiencing an arrhythmia state. As discussed above, if the rate signals fall below the detection threshold, the processor 26 simply continues to monitor subsequent atrial 300 and ventricular electrograms 304 to determine when an arrhythmia state is being experienced. However, if either of the rate signals is above the detection threshold, the processor 26 determines that the heart is experiencing an arrhythmia state and initiates a treatment protocol. For the present embodiment, the ventricular rate signal is first compared to the detection threshold.

After the processor 26 has detected an arrhythmia state (step 201), the processor 26 compares the atrial rate with the ventricular rate to ascertain whether the arrhythmia is determinable, such that the atrial rate is greater than the ventricular rate (step 202) or less than the ventricular rate (step 204), or not determinable, such that the atrial rate is equal to the ventricular rate (step 206).

If the atrial rate is greater than the ventricular rate (step 202), the processor 26 accesses the treatment look-up table 66 in database 34 to determine the treatment for this type of arrhythmia. After determining the type of treatment for this arrhythmia as being atrial therapy, the processor 26 communicates with the treatment centre 32 to administer this treatment protocol to the patient. The treatment centre 32 then sends signals to electrode 12a to administer the treatment protocol (step 208). After the electrode 12a administers the treatment, the electrodes 12 continue to sense and transmit heart signals (step 210).

If the processor 26 determines that the atrial rate is less than the ventricular rate (step 204), the processor 26 accesses the look-up table 66 in the database 34 to determine the treatment for this type of arrhythmia. After determining the type of treatment for this arrhythmia as being ventricular therapy/ventricular fibrillation therapy, the processor 26 communicates with the treatment centre 32 to administer this treatment protocol to the patient. The treatment centre 32 then sends signals to electrode 12b to administer the treatment protocol (step 212). After the electrode 12b administers the treatment, the electrodes 12 continue to sense and transmit heart signals (step 210).

If the processor 26 ascertains that the type of arrhythmia is not determinable, or is ambiguous i.e. the atrial rate equals the ventricular rate, further arrhythmia discrimination is required to determine the type of arrhythmia and whether treatment is necessary. A diagnostic test is then selected from the list of diagnostic tests 64 stored in the database 34 in order to produce stimulated heart signals (step 214). In this embodiment, the processor 26 determines from the list of tests 64 that monitoring the response of the heart to a paced atrial stimulation period shorter than the one detected will resolve the ambiguity. Accordingly, the processor 26 retrieves the measured atrial cycle length (CL) and ventricular CL when the arrhythmia was detected. The atrial CL is then used to determine stimulation for the treatment centre 32 to stimulate the patient's heart. After calculating the timing of the stimulation, as 50 ms less than the atrial cycle length, the processor 26 then signals the treatment centre 32 to stimulate the atrium 14 so that the atrium beats at the calculated timing as part of the diagnostic test. The treatment centre 32 then sends signals to electrode 12a corresponding to a pacing of the atrium 14 at the calculated timing (step 216).

During the pacing by electrode 12a to stimulate the atrium 14, both electrodes 12a and 12b continue to sense the heart signals, referred to as the stimulated heart signals, and transmit these signals to the I/O ports 22. The stimulated heart signals are then transmitted to the heart signal monitor 28 which receives the stimulated heart signals and translates the stimulated heart signals into a readable format. The formatted signals are then received by the processor 26 (step 218). The stimulated ventricular rate is then calculated by inverting the cycle length of successive stimulated ventricular electrograms, in the same manner discussed above, while the stimulated atrial rate is known due to the previously calculated timing of stimulation. The processor 26 then compares the ventricular rate to the detection threshold to determine if the stimulated ventricular rate falls below the detection threshold (step 220). If the stimulated ventricular rate falls below the detection threshold, the processor 26 determines that the detected arrhythmia does not require treatment and the apparatus 10 then continues to monitor heart signals to detect a subsequent arrhythmia state (step 210).

However, if the stimulated ventricular rate does not fall below the detection threshold, the processor 26 compares the stimulated ventricular rate with the predetermined stimulated atrial rate. If the two stimulated rates are equal (step 222), the processor 26 accesses the treatment look-up table 66 in database 34 to determine the treatment for this type of arrhythmia. The type of treatment (in this case, atrial therapy) is retrieved from the database 34 and the processor 26 then signals the treatment centre 32 to administer atrial therapy for the arrhythmia (step 224). The treatment centre 32 then sends signals to electrode 12a to administer the treatment. After the electrode 12a administers the treatment, the electrodes 12 continue to sense and transmit heart signals (step 210).

If the stimulated rates are not equal, the processor 26 compares the rates to determine if the stimulated ventricular rate is less than the stimulated atrial rate (step 226). The processor 26 then calculates a stimulated ventricular CL and determines if the stimulated ventricular CL is less than the ventricular CL (the ventricular CL at the time the arrhythmia was detected) (step 228) or if the stimulated ventricular CL is equal to the ventricular CL (step 230) by comparing the two values using the comparator 58. It will be understood that their values may be retrieved from the memory. If the stimulated ventricular CL and the ventricular CL are equal (step 230), the processor 26 accesses the treatment look-up table 66 in database 34 to determine the treatment for this type of arrhythmia. After determining the type of treatment for this arrhythmia as being ventricular therapy, the processor 26 communicates with the treatment centre 32 to administer this treatment to the patient. The treatment centre 32 then sends signals to electrode 12b to administer the treatment (step 232). After the electrode 12b administers the treatment, the electrodes 12 continue to sense and transmit heart signals (step 210).

Otherwise, if the stimulated ventricular CL is less than the ventricular CL (step 228), the processor 26 accesses the database 34 to determine the treatment for this type of arrhythmia. After determining the type of treatment for this arrhythmia as being atrial therapy, the processor 26 communicates with the treatment centre 32 to administer this treatment to the patient (step 234). The treatment centre 32 then sends signals to electrode 12a to administer the treatment. After the electrode 12a administers the treatment, the electrodes 12 continue to sense and transmit heart signals (step 210).

In another embodiment, as described with the first mode shown in FIG. 3, the electrodes 12 may be located on the skin surface (around the heart) of a patient to transmit heart signals to the I/O ports 22 (step 100). The electrodes 12 each transmit a set of heart signals to the heart signal monitor 28 representing the rates and shapes of the heart signals measured at their respective locations. It will be understood that the present embodiment eliminates the need for surgery to implant the electrodes 12 and the apparatus 10 within the patient's body.

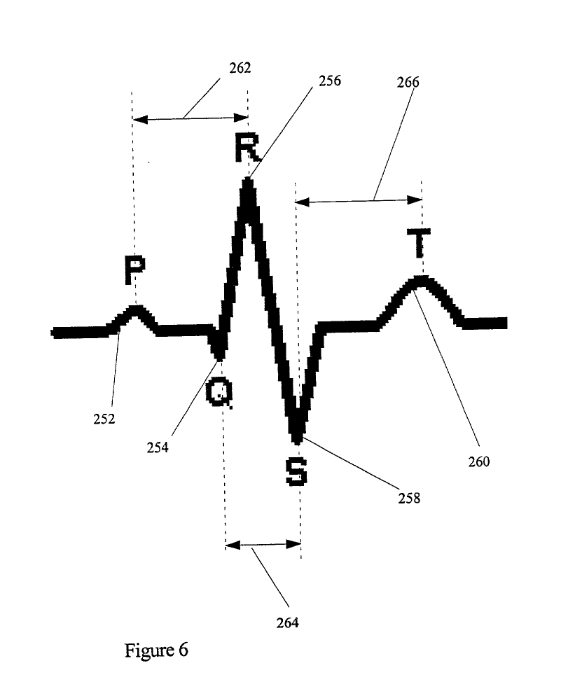

The heart signals are monitored (step 102) to determine when an arrhythmia state is being experienced. The heart signals are sensed by the electrodes 12 and transmitted to the heart signal monitor 28 via the I/O ports 22. The heart signals are then translated by the heart signal monitor 28 into a format which is readable by the processor 28 such as a cardiac cycle 250 as shown in FIG. 6.

As will be understood by one skilled in the art, a normal heart beat is caused by the sinoatrial node of the heart initiating an electrical impulse to depolarize the heart and to cause the heart to contract which in turn results in a cardiac cycle 250. The P wave 252, the Q wave 254, the R wave 256, the S wave 258 and the T wave 260 and the intervals between these waves represent different aspects of the cardiac cycle for the heart. The P wave 252 and the PR interval 262 indicate atrial depolarization with a normal PR interval being 0.12 to 0.2 seconds in length. The QRS complex 264 represents the spread of the electrical impulse through the ventricular muscle, or depolarization and generally lasts 0.08 to 0.12 seconds. The ST segment 266 and the T wave 260 represent the cardiac repolarization or recovery.

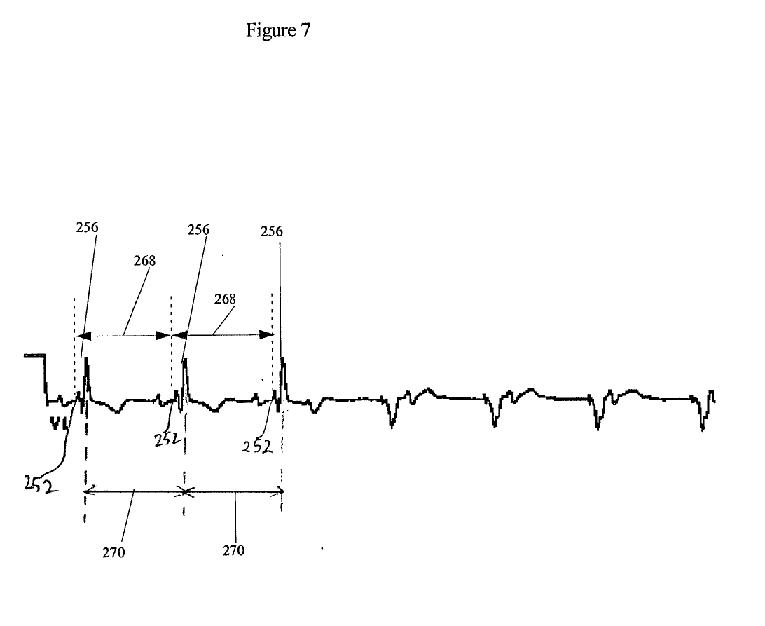

After receiving the formatted signals and storing the signals in memory 50, the CPU 52 retrieves a pair of rate signals by calculating the time elapsed between successive P waves 252 to determine an atrial cycle length 268 along with the time elapsed between successive R waves 256 to determine a ventricular cycle length 270 as shown in FIG. 7. The rate signals are then stored in the rate memory location 56. The rate signals corresponding to these two rates are then compared, using the comparator 58, to the pre-stored detection threshold 62 in order to determine if the heart is experiencing an arrhythmia state (step 104). As discussed above, if the rate signals fall below the detection threshold, the processor 26 simply continues to monitor subsequent cardiac cycles (step 102) to determine when an arrhythmia state is being experienced. However, if either of the rate signals is above the detection threshold, the processor 26 determines that the heart is experiencing an arrhythmia state.

After the processor 26 has detected an arrhythmia state, the processor 26 checks whether the arrhythmia is determinable or not determinable (step 106). This may be performed by comparing the atrial rate with the ventricular rate.

If the arrhythmia is determinable, the processor 26 accesses the treatment look-up table 66 in database 34 to determine the treatment for this type of arrhythmia and to adminster the treatment (step 108). After administering the treatment, the apparatus returns to a heart signal sensing state (step 109).

If the processor 26 ascertains that the type of arrhythmia is not determinable, or is ambiguous i.e. the atrial rate equals the ventricular rate, further arrhythmia discrimination is required to determine the type of arrhythmia and whether treatment is necessary. A diagnostic test is then selected from the list of diagnostic tests 64 stored in the database 34 in order to produce stimulated heart signals (step 110). After selecting the diagnostic test, the heart is stimulated (step 112) according to the parameters of the selected test. This stimulation may be via, for example, a mild shock to the heart, vagal stimulation or drug infusion.

After the heart has been stimulated, the electrodes 12 continue to sense the heart signals, referred to as the stimulated heart signals, and transmit these signals to the IO ports 22 (step 114). The stimulated heart signals are then transmitted to the heart signal monitor 28 which receives the stimulated heart signals and translates the stimulated heart signals into a cardiac cycle. The formatted signals are then received by the processor 26 which then extracts the rate signals for the atrial and ventricular stimulated cycle lengths in order to calculate stimulated atrial and ventricular rates.

The stimulated atrial and ventricular rates are then monitored to determine the type of arrhythmia being experienced by the heart (step 116). After determining the type of arrhythmia, a check is performed to see whether or not the arrhythmia requires treatment (step 118). If the arrhythmia requires treatment, the treatment is identified and administered (step 108) and if not, the apparatus simply returns to a heart signal sensing state (step 109).

By stimulating the patient's heart after an arrhythmia state has been detected to produce stimulated heart signals and monitoring these stimulated heart signals, arrhythmias which do not require treatment may be more readily detected and the number of unnecessary treatments reduced. As described above, each time treatment is administered to a patient to treat arrhythmia, the patient experiences pain and discomfort. Therefore, a reduction in the number of unnecessary treatments provided to a patient may be realized by determining whether treatment is required each time an arrhythmia state is detected. As well, arrhythmias which were previously not discriminated, may be discriminated. Instead of simply providing treatment by default to those arrhythmias which could not be determined, the steps of stimulating the heart to create stimulated heart signals and monitoring of these signals may assist in discriminating the type of arrhythmia being experienced. Furthermore, more appropriate treatments are administered to the patient once arrhythmia is detected.

Alternatively, the list of diagnostic tests may be stored within the processor 26 so that the time required to select the diagnostic test is reduced.

In another embodiment, in order to distinguish between heart signals and stimulated heart signals, the processor 26 may simply set a flag once the processor 26 has communicated the diagnostic test to the treatment centre 32 such that all heart signals received subsequent to the diagnostic test are seen as stimulated heart signals. Furthermore, if the diagnostic test causes the cycle length of the heart rate to be shortened, the expected cycle length of the retrieved stimulated rate signals may be used to identify that stimulated heart signals are being read.

It will be understood that other information such as shape and morphology of the heart signals may also be retrieved from the formatted signals and used to determine the type of arrhythmia being experienced by the patient by comparing these signals to predetermined morphology or shape signals stored in the predetermined stored values 60.

It will also be understood that the parameters of the detection threshold 62, the treatment look-up table 66 or the list of diagnostic tests may be changed.

Furthermore, although pacing has been described as the method of stimulating the heart, other methods may also be implemented such as vagal stimulation or drug infusion.

The different treatments which are available for treating arrhythmia will be well known to those skilled in the art and is not restricted to those listed above. It will be understood that pacing of the heart may include subthreshold pacing, burst pacing, ultra-high frequency pacing, 60 cycle or n times 60 cycle pacing, extrastimulation, rapid overdrive pacing of the atrium or ventricle, vagal stimulation and high frequency stimulation.

Although the invention has been described with reference to certain specific embodiments, various modifications thereof will be apparent to those skilled in the art without departing from the spirit and scope of the invention as outlined in the claims appended hereto.

The embodiments of the invention in which an exclusive property or privilege is claimed are defined as follows:

1. A method of providing arrhythmia discrimination comprising the steps of:
receiving signals from a heart;
monitoring said signals to detect a condition indicative of at least one of a number arrhythmia states;
detecting said arrhythmia state;
performing a diagnostic test on said heart to produce stimulated heart signals;
monitoring said stimulated heart signals to determine which of said states is detected.

2. The method of claim 1 wherein said step of performing said diagnostic test comprises the steps of:
selecting a diagnostic test corresponding to one of said arrhythmia states;
calculating a level of stimulation for said heart; and
stimulating said heart at said level of stimulation.

3. The method of claim 2 wherein said step of stimulating said heart is selected from the group comprising of pacing, vagal stimulation and drug infusion.

4. The method of claim 1 wherein said step of calculating a level of stimulation for said heart comprises the steps of:
calculating a cycle length of said heart rate; and
subtracting a predetermined value from said cycle length to produce a new cycle length.

5. The method of claim 1 wherein said step of monitoring said heart signals comprises the steps of:
retrieving rate signals from said heart signals; and
determining when said rate signals exceed a detection threshold.

6. The method of claim 1 wherein said step of monitoring said stimulated heart signals comprises the steps of:
retrieving stimulated signals from said stimulated heart signals;
comparing said stimulated signals with predetermined criteria to determine said arrhythmia.

7. The method of claim 6 wherein said stimulated signals are stimulated rate signals or morphology signals.

8. The method of claim 1 further comprising the step of comparing said arrhythmia with a list of target arrhythmias and treatments to determine treatment for said arrhythmia.

9. The method of claim 1 wherein said heart signals comprise atrial and ventricular heart signals.

10. Apparatus for providing arrhythmia discrimination comprising:
a set of electrodes for sensing and transmitting signals from a heart;
a heart signal monitor for receiving said signals and for translating said signals to processor-readable signals;
a processor for retrieving rate signals from said processor-readable heart signals to detect a condition indicative of an arrhythmia state based on said rate signals and for selecting a diagnostic test when said arrhythmia is detected;
wherein said diagnostic test causes said heart to produce stimulated heart signals which are sensed and transmitted by said set of electrodes to said heart signal monitor for translating to processor-readable stimulated heart signals; and
wherein said processor retrieves stimulated signals from said processor-readable stimulated heart signals and compares said stimulated signals with predetermined criteria to discriminate said arrhythmia state.

11. The apparatus of claim 10 further comprising a treatment centre for treating said arrhythmia.

12. The apparatus of claim 10 further comprises a database for storing treatments associated with a list of arrhythmias wherein after said arrhythmia has been discriminated, said processor accesses said database to compare said arrhythmia with said list of arrhythmias to determine treatment for said arrhythmia.

13. The apparatus of claim 10 wherein stimulated signals are stimulated rate signals or stimulated morphology signals.
